(19) United States
(12) Patent Application Publication
(10) Pub. No.: US 2020/0229978 A1
(43) Pub. Date: Jul. 23, 2020

(54) ENHANCED DRAINAGE OF FAILED GLAUCOMA DRAINAGE DEVICE (GDD)

(71) Applicant: UNIVERSITY OF FLORIDA RESEARCH FOUNDATION, INCORPORATED, Gainesville, FL (US)

(72) Inventors: Mark B. SHERWOOD, Gainesville, FL (US); Craig A. MEYERS, Gainesville, FL (US)

(21) Appl. No.: 16/634,360

(22) PCT Filed: Aug. 9, 2018
(86) PCT No.: PCT/US2018/046043

§ 371 Date: Jan. 27, 2020

Related U.S. Application Data
(60) Provisional application No. 62/544,367, filed on Aug. 11, 2017.

(51) Int. Cl. A61F 9/007; A61F 2/82; A61K 31/407; A61K 9/00

(52) U.S. Cl. CPC A61F 9/00781; A61F 2/82; A61K 9/0048; A61K 9/0019; A61K 31/407

(57) ABSTRACT

Example embodiments provide techniques for managing and resolving failed glaucoma drainage devices (GDDs). In example embodiments, these techniques require only a simple office or minor-operating room procedure. For example, a GDD may fail due to a fibrous capsule forming about the plate portion of the GDD and preventing aqueous flowing through the tube of the GDD from the anterior region from leaving the encapsulated bleb area. The failed GDD may be made to function effectively again, in an example embodiment, by placing and/or implanting a stent implant into the wall of the fibrous capsule to allow aqueous to flow through the stent into the subconjunctival space. According to an aspect of the present invention, a method for treating a failed GDD is provided. In an example embodiment, the method comprises placing a stent implant in the wall of the capsule surrounding a posteriorly placed episcleral plate of the GDD.

### TECHNICAL FIELD

Embodiments of the present invention relate to the drainage of failed glaucoma drainage devices (GDDs). Example embodiments provide techniques for managing and resolving failed GDDs in this respect and otherwise.

### BACKGROUND

Approximately 15-40% of human glaucoma drainage device (GDD) implants fail to moderate an acceptable intraocular pressure (IOP) over time. For example, the GDD fails to provide sufficient drainage over an extended period of time. Currently, treatment for failed GDDs consists of either laser cyclophotocoagulation or placing a second GDD implant. Laser cyclophotocoagulation us a cyclodestructive procedure that carries the risk of long term inflammation and, if excessively done, can result in phthisis and blindness. Current therapies in cases of failed glaucoma drainage implants include (a) an additional GDD, which takes considerable time in the operating room and may not be effective in the long term, (b) a cyclodestructive procedure which may be effective but carries the risk of long term inflammation and, if excessively done, can result in phthisis and blindness, or (c) needling the bleb over the plate which is generally ineffective in the long term as the needle track generally scars closed within a few weeks.

Therefore, there is a need for improved methods of managing and resolving failed GDDs.

### BRIEF SUMMARY

Example embodiments provide techniques for managing and resolving failed GDDs. In example embodiments, these techniques require only a simple office or minor-operating room procedure. In an example embodiment, a GDD has failed due to a fibrous capsule forming about the plate portion of the GDD and preventing the aqueous flowing through the tube of the GDD from the anterior chamber region from leaving the encapsulated bleb area. The failed GDD may be made to function effectively again, in an example embodiment, by placing and/or implanting a stent implant into the wall of the fibrous capsule to allow aqueous to flow through the stent into the subconjunctival space.

According to an aspect of the present invention, a method for treating a failed GDD is provided. In an example embodiment, the method comprises placing a stent implant in the wall of the capsule surrounding a posteriorly placed episcleral plate of the GDD.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a partial cross-section diagram of a failed GDD implant having a subconjunctivally placed tube implant, according to an example embodiment of the present invention;

FIG. 2 is a perspective diagram of a failed GDD implant having a subconjunctivally placed tube implant, according to an example embodiment of the present invention; and

FIG. 3 provides a flowchart of processes and/or procedures for treating a failed GDD implant, in accordance with an example embodiment.

### DETAILED DESCRIPTION OF SOME EXAMPLE EMBODIMENTS

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. The term “or” (also denoted “/”) is used herein in both the alternative and conjunctive sense, unless otherwise indicated. The terms “illustrative” and “exemplary” are used to be examples with no indication of quality level. The terms “generally” and “approximately” refer to within engineering and/or manufacturing limits, unless otherwise indicated. Like numbers refer to like elements throughout.

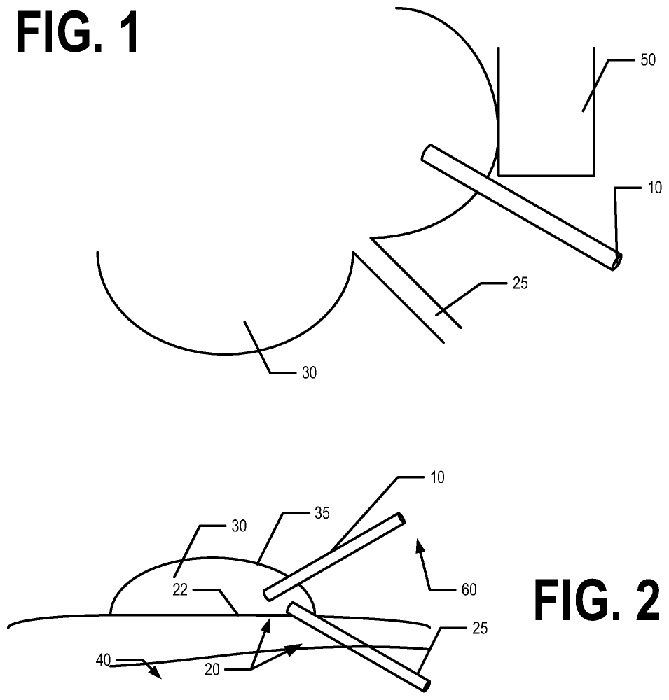

FIGS. 1 and 2 provide views of a failed glaucoma drainage device (GDD) 20 that has been made functional again though the placement of a stent implant 10 in the subconjunctival space 60. For example, the GDD 20 may comprise a posteriorly placed episcleral plate (e.g., a plate portion 22). For example, the GDD 20 may have failed because a fibrous capsule 30 formed over the plate portion 22 of the GDD 20. For example, the GDD 20 comprises a tube 25 that extends from the plate portion 22 of the GDD 20 into the anterior chamber 40 of the patient's eye. The GDD 20 is configured to permit aqueous to flow out from the anterior chamber 40 to reduce pressure therein. However, the formation of the fibrous capsule 30 on the plate portion 22 of the GDD 20 reduces the flow of aqueous through the GDD 20 and/or prevents aqueous from flowing through the GDD 20. In an example embodiment, a failed GDD is a GDD 20 which is not sufficiently lowering the intraocular pressure due to the presence of too thick a fibrous capsule 30 formed about the plate area of the GDD 20. The stent implant 10 is placed in the capsular wall 35 of the fibrous capsule 30 such that aqueous can flow through the GDD 20 and drain through the capsule 30 via the open internal bore of the stent implant 10. In an example embodiment, the stent implant 10 is configured to drain aqueous into the subconjunctival space 60 between or in front of the muscles 50 attached to the patient's eye.

For example, in an example embodiment, the stent implant 10 allows aqueous which is coming up the tube 25 of the failed GDD 20 to escape a localized bleb area of the plate portion 22 and spread to the adjacent quadrant. In various embodiments, the stent implant 10 is one of a Xen45, Xen63, or ExPress implant or a similar implant. In various embodiments, the length of the stent implant 10 is in the range of 3 to 10 mm. In an example embodiment, the stent implant 10 is an approximately 6 mm long tube. For example, the stent implant 10 may be a hollow cylindrical tube with a wall that defines an external diameter and an inside diameter. In an example embodiment, the stent implant 10 has an external diameter of approximately 150 um such that the stent implant 10 may be injected using a 27-gauge needle. In various embodiments, the stent implant has an external diameter of approximately 100 to 200 um. In an example embodiment, the inside diameter of the tube of the stent implant 10 is in the range of 30 to 100 um. For example, the inside diameter of the tube of the stent implant 10 is 45 um in an example embodiment and 63 um in another example embodiment. In an example embodiment, the stent implant 10 may be made of stainless steel, silicone, Poly(methyl methacrylate) (PMMA), porcine gelatin cross-linked with glutaraldehyde, (Poly Styrene-block-IsoButylene-block-Styrene or SIBS) and/or implant appropriate material.

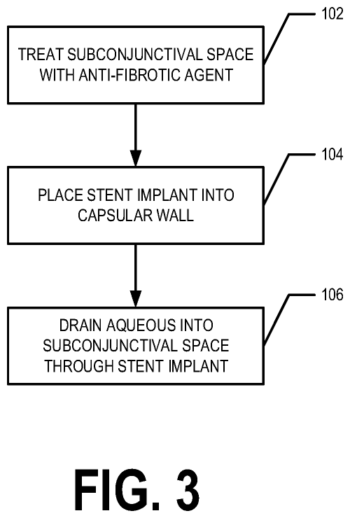

FIG. 3 provides a flowchart showing processes and/or procedures for the use of a stent implant 10 to allow a failed GDD 20 to effectively mediate the intraocular pressure within the patient's eye. Starting at block 102, in an example embodiment, prior to the implantation and/or placement of the stent implant 10 an anti-fibrotic agent may be injected into the subconjunctival space 60. For example, a few hours to multiple days prior to the implantation and/or placement of the stent implant 10, an anti-fibrotic agent may be injected into the subconjunctival space 60. In an example embodiment, the anti-fibrotic agent is mitomycin-c. In an example embodiment, about 0.1 ml of mitomycin-c is injected subconjunctivally into the area adjacent to the encapsulated plate 22. In an example embodiment, after injecting the anti-fibrotic agent into the subconjunctival space, the anti-fibrotic agent is spread within the subconjunctival space 60 via a gentle massage of the overlying conjunctival tissue using, for example, a q-tip cotton applicator.

Sometime later (e.g., a few hours to a few days later), at block 104, the stent implant 10 is injected through the wall 35 of the capsule 30 overlying the episcleral plate (e.g., the plate portion 22) (which is usually placed in the superotemporal quadrant and is generally visible beneath the conjunctiva when the patient looks down). For example, the stent implant 10 may be injected through the wall 35 of the capsule 30 via a short subconjunctival tract from the adjacent superonasal or perhaps inferotemporal quadrant. In an example embodiment, the implant is injected using a 27-gauge dual-beveled needle. This would allow the aqueous which is coming up the tube 25 to escape the localized bleb area of the plate 22 and spread to the adjacent quadrant, at block 106.

Generally, the stent implant 10 may be implanted and/or placed as an office procedure or minor-op room under topical anesthesia (similar to current trabeculectomy bleb needling techniques) and there is no risk of hitting any other structures as the plastic of the plate 22 underlies the encapsulated bleb or fibrous capsule 30. The procedure should only take a few minutes and is expected to provide cost savings from current therapies. Thus, various embodiments provide an improved patient experience and improved treatment results.

These techniques may be used for treating failed GDDs 20 in humans and also in the Veterinary arena for dogs, for example, with failed, encapsulated GDDs 20. This occurs in an even higher percentage of cases in dogs than in humans—approaching 70-100% of cases. This technique has been used in a dog with high intraocular pressure and bleb encapsulation of a GDD 20 using mitomycin-c and a stent implant 10 of the ExPress implant type and at 16 months post-procedure the dog's intraocular pressure remains in the low normal range.

Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for treating a failed glaucoma drainage device (GDD), the method comprising:
placing a stent implant in the wall of the capsule surrounding a posteriorly placed episcleral plate of the GDD.

2. The method of claim 1, further comprising injecting an anti-fibrotic agent subconjunctivally into the area adjacent to the plate before placing the stent implant.

3. The method of claim 2, wherein the anti-fibrotic agent is mitomycin-c.

4. The method of claim 3, wherein about 0.1 ml of mitomycin-c is injected.

5. The method of claim 2 further comprising, after injecting the anti-fibrotic agent, spreading the anti-fibrotic agent by gently massaging with an applicator.

6. The method of claim 1, wherein the stent implant allows aqueous which is coming up the tube of the failed GDD to escape a localized bleb area of the plate and spread to the adjacent quadrant.

7. The method of claim 1, wherein a failed GDD is a GDD which is not sufficiently mediating the intraocular pressure due to the presence of a fibrous capsule formed about the plate area of the GDD.

8. The method of claim 1, wherein placing the stent implant comprises injecting the stent implant through the wall of the capsule.

9. The method of claim 8, wherein the stent implant is injected via a short subconjunctival tract from the adjacent supernasal.

10. The method of claim 8, wherein the stent implant is injected via the inferotemporal quadrant.

11. The method of claim 8, wherein the stent implant is injected using a 27-gauge needle.

12. The method of claim 1, further comprising, prior to placing the stent implant, administering local anesthesia.

13. The method of claim 1, wherein the placing of the stent implant is performed as an office procedure or minor-operation room procedure.

14. The method of claim 1, wherein the stent implant is tube having a length that is in a range of 3 to 10 mm.

15. The method of claim 1, wherein a patient having the failed GDD is a human or a dog.

16. An implant assembly comprising:
a glaucoma drainage device (GDD) comprising a plate portion and a tube, the plate portion being encapsulated by a fibrous capsule; and
a stent implant placed in a wall of the fibrous capsule.

17. The implant assembly of claim 16, wherein the stent implant allows aqueous to flow from inside the fibrous capsule to a subconjunctival space of a patient's eye.

18. The implant assembly of claim 16, wherein the tube allows aqueous to flow from inside an anterior chamber of a patient's eye to the interior of the fibrous capsule.

19. The implant assembly of claim 16, wherein the fibrous capsule impedes the flow of aqueous away from the plate portion.

20. The implant assembly of claim 16, wherein the stent implant is tube having a length that is in a range of 3 to 10 mm.
